(19) United States
(12) Patent Application Publication
(10) Pub. No.: US 2014/0025104 A1
(43) Pub. Date: Jan. 23, 2014

(54) BIOLOGICAL ADHESIVE SHEET AND DEVICE FOR ATTACHING BIOLOGICAL ADHESIVE SHEET

(71) Applicant: TERUMO KABUSHIKI KAISHA, Shibuya-ku (JP)

(72) Inventor: Naoki Ishii, Kanagawa (JP)

(73) Assignee: TERUMO KABUSHIKI KAISHA, Shibuya-ku (JP)

(21) Appl. No.: 14/037,089

(22) Filed: Sep. 25, 2013

Related U.S. Application Data
(63) Continuation of application No. PCT/JP2012/050220, filed on Jan. 10, 2012.

(30) Foreign Application Priority Data
Mar. 30, 2011 (JP) 2011-073862

(51) Int. Cl. A61B 17/00

(52) U.S. Cl. CPC A61B 17/0057
USPC 606/213

(57) ABSTRACT

A highly safe biological adhesive sheet capable of exerting a strong adhesive force almost without requiring a pressing force when the biological adhesive sheet is attached to living tissue. The biological adhesive sheet includes a substrate in which a plurality of through-holes are formed; and an adhesive section in which a plurality of protrusions that protrude on one surface side of the substrate are formed, wherein the protrusions make contact with living tissue and are made to adhere to the living tissue by a Van der Waals' forces by drawing the living tissue from the other surface side of the substrate through the through-holes.

### CROSS REFERENCES TO RELATED APPLICATIONS

This application is a continuation of International Application No. PCT/JP2012/050220 filed on Jan. 10, 2012, and claims priority to Japanese Application No. 2011-073862 filed on Mar. 30, 2011, the entire content of both of which is incorporated herein by reference.

### TECHNICAL FIELD

The present invention generally relates to a biological adhesive sheet capable of adhering to living tissue and a device for attaching the biological adhesive sheet to living tissue.

### BACKGROUND DISCUSSION

A method is known for treatment of pneumothorax, which occurs when an air leakage part is formed due to a tear of a cyst on a lung and inspired air leaks from the air leakage part to a lung cavity. This causes the gas in the lung cavity to compress the lung to preclude the lung from taking in the external air. In the known method, a PGA (polyglycolic acid) sheet to which a fibrin glue is applied is attached to the air leakage part.

Furthermore, as a treatment for dilated cardiomyopathy, which is a heart disease in which the heart cell changes and the myocardium stretches, there has been proposed a method in which a cell sheet made by culturing the patient's muscle is attached from outside of the left ventricle to thereby suppress the stretch of the myocardium (refer to e.g. Japanese Patent Laid-open No. 2011-4750).

However, living tissue is very delicate. Therefore, it is preferable to apply as small pressing force as possible to avoid damage to the living tissue when the above-described sheet is attached but, if the pressing force is too small, the adhesive force of the sheet may be lowered.

### SUMMARY

The disclosure herein provides a highly safe biological adhesive sheet capable of exerting a strong adhesive force almost without requiring a pressing operation when the biological adhesive sheet is attached to living tissue, and provides a device for attaching the biological adhesive sheet.

A biological adhesive sheet according to an exemplary embodiment of the disclosure herein includes a substrate in which a plurality of through-holes are formed and an adhesive section in which a plurality of protrusions are formed that protrude from one surface side of the substrate. The protrusions make contact with living tissue and are made to adhere to the living tissue by the Van der Waals' forces by drawing the living tissue from the other surface side of the substrate through the through-holes.

A device for attaching the biological adhesive sheet according to an exemplary embodiment of the disclosure includes a holding section that holds a biological adhesive sheet in which through-holes are formed and brings an adhesive section of the biological adhesive sheet into contact with living tissue. The holding section has a suction part supplied with a negative pressure, thus allowing suction of the living tissue through the through-holes of the biological adhesive sheet. The suction part draws (sucks) the living tissue through the through-holes of the biological adhesive sheet to bring the adhesive section of the biological adhesive sheet into contact with the living tissue and thereby cause the adhesive section to adhere to the living tissue.

In the biological adhesive sheet configured in the above-described manner, the protrusions make contact with living tissue and are made to adhere to the living tissue by the Van der Waals' forces drawing the living tissue from the other surface side of the substrate through the through-holes. Therefore, a strong adhesive force can be exerted without requiring pressing when the biological adhesive sheet is attached to the living tissue, and safety is enhanced.

If the number of the protrusions per 100 μm² is at least one and the protrusions have a length of 1 μm to 50 μm and a maximum outer diameter of 5 nm to 10 μm, the protrusions can be made to favorably adhere to the living tissue by utilizing the Van der Waals' forces.

If the adhesive section is partially formed on the substrate, a variety of designs are allowed depending on the application site of the living tissue and the purpose of use.

If the adhesive section has a projecting base that is formed so as to protrude from an outer surface of the substrate and has a protrusion-formed surface inclined to the outer surface and the protrusions are formed on the protrusion-formed surface, when the inclined protrusion-formed surface is separated from the living tissue, it gets detached from one side. Thus, the protrusions formed on the protrusion-formed surface can be easily detached from the living tissue.

If the porosity of the through-holes is 45% to 85%, the living tissue can be favorably drawn (sucked) through the through-holes.

In the device for attaching the biological adhesive sheet configured in the above-described manner, the suction part draws the living tissue through the through-holes of the biological adhesive sheet to bring the adhesive section of the biological adhesive sheet into contact with the living tissue and cause the adhesive section to adhere to the living tissue. Therefore, a strong adhesive force can be exerted without requiring pressing when the biological adhesive sheet is attached to the living tissue, and safety is enhanced.

If the suction part is partially formed at a site in the holding section with which the biological adhesive sheet makes contact, only the site requiring suction can be drawn (sucked) depending on the application site of the living tissue and the purpose of use.

### BRIEF DESCRIPTION OF THE DRAWINGS

The accompanying drawings are included in the specification and form a part of the disclosure here, and are used to disclose aspects and principles of the disclosure here together with the detailed description set forth below.

FIG. 1 is a plan view showing a biological adhesive sheet according to an exemplary embodiment of the disclosure.

FIG. 2 is a sectional view along line 2-2 in FIG. 1.

FIG. 3 is a partially enlarged perspective view showing part of an adhesive section of the biological adhesive sheet according to the exemplary embodiment.

FIG. 4 is a sectional view showing protrusions of the biological adhesive sheet according to the exemplary embodiment.

FIG. 5 is a sectional view showing a modified example of the protrusions of the biological adhesive sheet according to the exemplary embodiment.

FIG. 6 is a partially enlarged sectional view showing a mold for manufacturing the protrusions.

FIG. 7 is a partially enlarged sectional view showing a state in which a material is poured into the mold.

FIG. 8 is a partially enlarged sectional view showing removal of the protrusions from the mold.

FIG. 9 is a plan view showing a device for attaching the biological adhesive sheet according to an exemplary embodiment.

FIG. 10 is a side view showing attachment of the biological adhesive sheet using an exemplary embodiment of the device for attaching the biological adhesive sheet.

FIG. 11 is a side view showing a state in which part of the biological adhesive sheet is brought into contact with living tissue by the exemplary embodiment of the device for attaching the biological adhesive sheet.

FIG. 12 is a partially enlarged side view showing how projecting bases of the biological adhesive sheet make contact with the living tissue.

FIG. 13 is a side view showing a state in which the biological adhesive sheet is attached to the living tissue by the exemplary embodiment of the device for attaching the biological adhesive sheet.

FIG. 14 is a partially enlarged sectional view showing a state in which the protrusions of the biological adhesive sheet adhere to the living tissue.

FIG. 15 is a side view showing peeling of the biological adhesive sheet from the living tissue by the exemplary embodiment of the device for attaching the biological adhesive sheet.

FIG. 16 is a partially enlarged side view showing how the projecting bases of the biological adhesive sheet get detached from the living tissue.

FIG. 17 is a sectional view showing another exemplary embodiment of the biological adhesive sheet.

### DETAILED DESCRIPTION

Exemplary embodiments (embodiments described as example of the biological adhesive sheet disclosed here) of the disclosure will be described below with reference to the drawings. In some cases, the dimensional ratio of the drawing is different from the actual ratio due to exaggeration for convenience of explanation.

A biological adhesive sheet 100 according to one exemplary embodiment of the disclosure herein is a sheet attached to living tissue M. Examples of the living tissue M to which the biological adhesive sheet 100 is made to adhere include, but are not limited to, the lung, heart, trachea, and esophagus. The adhesion target is not particularly limited as long as it is living tissue. As an example in which the biological adhesive sheet 100 is attached to a lung, for example, it will be possible to attach it from the outside in order to block an air leakage part as a treatment for pneumothorax. As an example in which the biological adhesive sheet 100 is attached to a heart, for example, it will be possible to attach it to the left ventricle from the outside as a treatment for dilated cardiomyopathy.

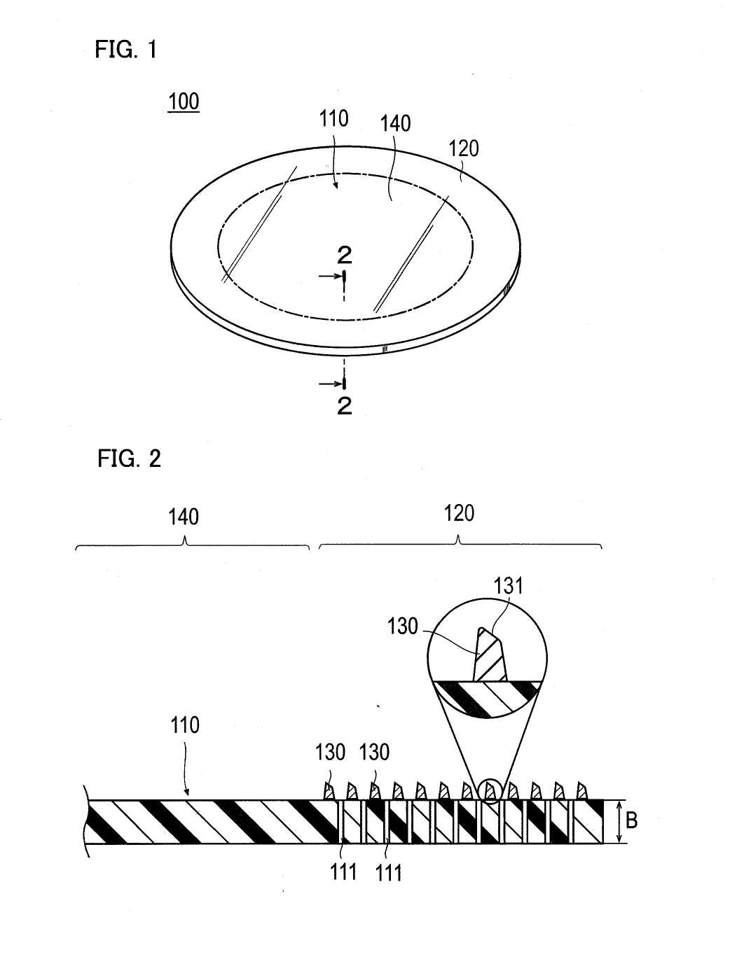

As shown in FIG. 1, the biological adhesive sheet 100 includes a substrate 110 with a flat plate shape. An adhesive section 120 that exerts an adhesive force to living tissue is formed at the rim part of the substrate 110. A flat surface section 140 that does not exert an adhesive force to living tissue is formed so as to be surrounded by the adhesive section 120.

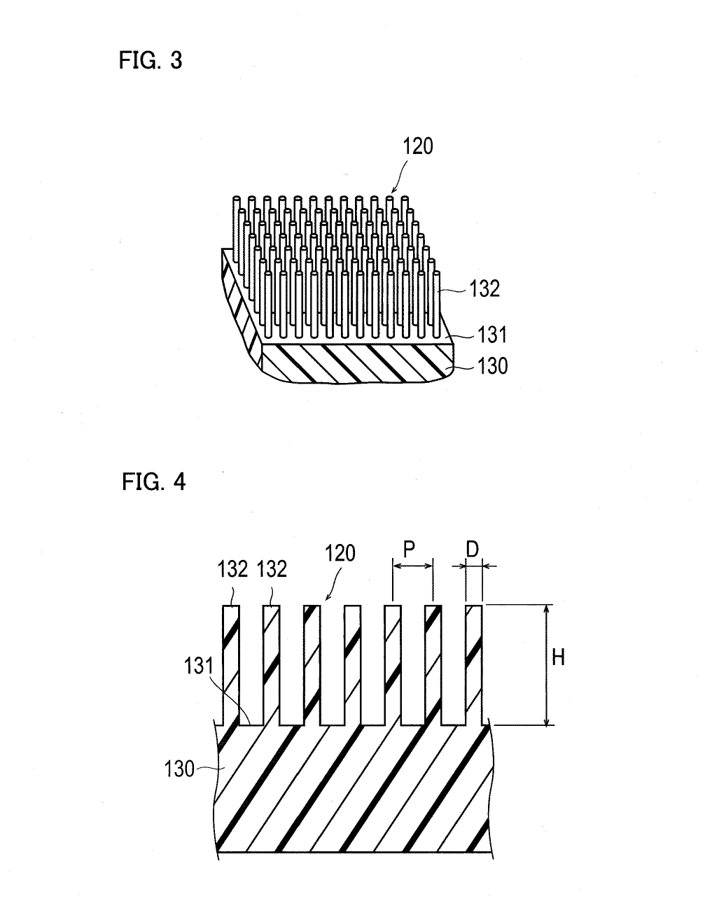

As shown in FIG. 2, in the adhesive section 120, projecting bases 130 are formed that protrude from one surface of the substrate 110. At the upper part of the projecting base 130, a protrusion-formed surface 131 inclined to the outer surface of the substrate 110 is formed. The protrusion-formed surface 131 is formed so as to be inclined and become higher relative to the outer surface of the substrate in the direction from one side of the biological adhesive sheet 100 to the other side. As shown in FIGS. 3 and 4, on this protrusion-formed surface 131, plural minute protrusions 132, on the nanometer order, are formed so as to protrude therefrom.

Furthermore, in the substrate 110, plural through-holes 111 are formed in the area where the adhesive section 120 is formed. The porosity of the through-holes 111 in the adhesive section 120 (ratio of the area of the holes to the surface area) is approximately 45% to 85%, for example, and preferably 50% to 70%, although not particularly limited as long as the living tissue M can be drawn (sucked) from the opposite surface through the through-holes 111.

When the adhesive section 120 in which the minute protrusions 132 are formed is brought into tight contact with the living tissue M and pressed, the adhesive state can be maintained by utilizing the Van der Waals' forces between the minute protrusions 132 and the living tissue M without using an additional adhesive. That is, by providing the plural minute protrusions 132 to increase the surface area of the adhesive section 120, the Van der Waals' forces can be generated with such magnitude that an adhesion state with the adhesion target can be kept. This adhesion function is exerted not only in a gas but also in a liquid although the Van der Waals' forces decreases. A known structure that allows adhesion by utilizing the Van der Waals' forces is a micro-fibrous structure as seen, for example, in the bottom of the foot of the gecko.

Furthermore, the protrusions 132 are formed on the protrusion-formed surface 131 at an inclination relative to the surface of the substrate 110. Therefore, the biological adhesive sheet 100 can be easily peeled off by peeling the adhesive section 120 from one side of the biological adhesive sheet 100 (lower side of the protrusion-formed surface 131). Thus, it is possible to peel off the biological adhesive sheet 100 and attach it again when it is not attached to a desirable position.

It is preferable that the thickness B of the substrate 110 is arbitrarily designed depending on the application site of the living tissue M and the purpose of use. The thickness B is, for example, 3 μm to 3000 μm and preferably 30 μm to 300 μm.

The inclination angle of the protrusion-formed surface 131 of the projecting base 130 relative to the outer surface of the substrate 110 is, for example, 5 to 45° and preferably 20 to 30°, although arbitrarily set and not particularly limited. The height of the projecting base 130 is, for example, 1 to 50 μm and preferably 10 to 30 μm, although arbitrarily set and not particularly limited. The area of one protrusion-formed surface 131 is, for example, 1 μm² to 50 μm² and preferably 10 μm² to 25 μm², although arbitrarily set and not particularly limited. One to 10⁶ protrusions 132 are formed per 100 μm² and, more preferably, 20 to 30 protrusions 132 are formed per 1 μm².

The arrangement pattern of the projecting bases 130 is not particularly limited. That is, they may be irregularly disposed although illustrated as regularly disposed in the exemplary embodiment.

The protrusion 132 is formed with a column shape (circular column shape in the exemplary embodiment). The maximum outer diameter D of the protrusion 132 is 5 nm to 10 μm and preferably 0.1 μm to 0.5 μm. The height H of the protrusion 132 is 1 μm to 50 μm and preferably 10 μm to 50 μm. The pitch P of the protrusion 132 is 0 μm to 1 μm and preferably 0.05 μm to 0.5 μm. The maximum outer diameter D represents the length of the longest part in the section perpendicular to the extension direction (protrusion direction) of the protrusion 132 and can be used even when the section does not necessarily have a circular shape.

One or more protrusions 132 are formed per 100 μm² and preferably 50 or more protrusions 132 are formed per 100 μm². If the protrusion 132 has the above-described shape and dimensions, an adhesive force can be exerted by the Van der Waals' forces in either a gas or a liquid medium.

The structure for adhesion is not limited to the structure that allows adhesion by the Van der Waals' forces by utilizing the minute protrusions 132. For example, a sticking agent (adhesive) that exerts a sticking force (adhesive force) in a solvent including water may be applied on the adhesion part. Examples of such a sticking agent (adhesive) include 3,4-dihydroxy-L-phenylalanine (dopamine, DOPA), which is an adhesive peptide having a catechol group, derivatives thereof, and polymers and copolymers of them. Furthermore, polysaccharides such as dextran, dextrin, and derivatives of them may be applied.

The arrangement pattern of the protrusions 132 is not particularly limited. They may be irregularly disposed although being regularly disposed in a lattice manner in the exemplary embodiment.

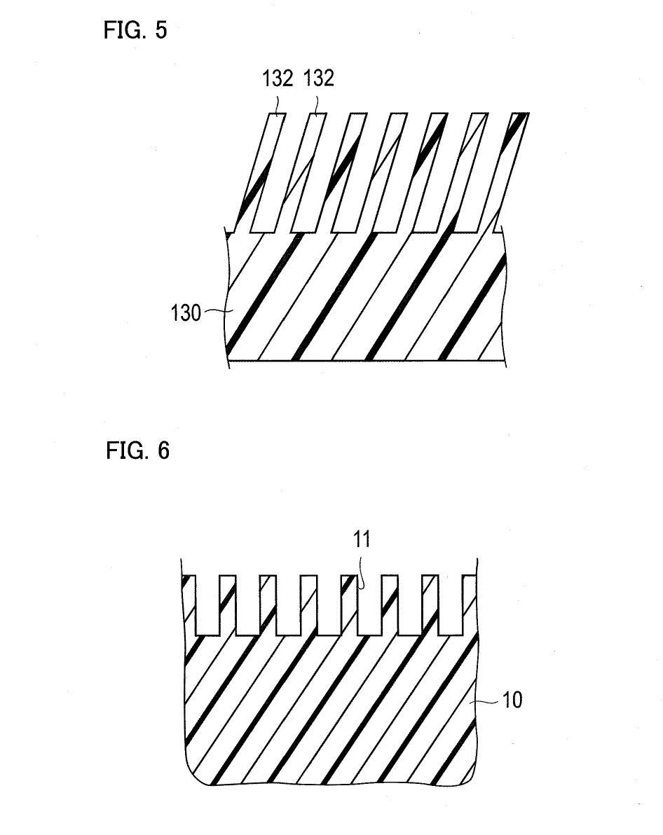

In the exemplary embodiment, the protrusion 132 is formed so as to extend perpendicularly from the protrusion-formed surface 131. However, like in another exemplary embodiment as shown in FIG. 5, it may be formed with an inclination to the protrusion-formed surface 131. The inclination angle X can be set from 0° to 60° and, preferably, 0° to 30°. The inclination direction and inclination angle may be made different by each protrusion 132.

The shape of the protrusion 132 is not limited to the circular column shape and may be, for example, a column shape whose section is a polygonal shape. Furthermore, the protrusion 132 does not necessarily need to have the same section in its whole body from the base end part joined to the substrate 110 to the tip part. For example, it is also possible that the section of the tip part is set larger or smaller than that of the base end part.

The projecting base 130 may be formed monolithically with the substrate 110 or may be formed by joining an additional member to the outer surface of the substrate 110 by bonding or the like.

As the constituent material of the substrate 110, the projecting base 130, and the protrusion 132, one having biocompatibility and flexibility is preferable. Examples of an acceptable material include, but are not limited to, silicon-based resins, polyurethane resins, and poly(meth)acrylate resins. Furthermore, a biologically active agent such as an immunosuppressant and/or an anti-cancer agent may be contained in at least one of the substrate 110, the projecting base 130, and the protrusion 132.

The biologically active agent is not particularly limited as long as it is a substance that acts on the living tissue M. Examples of a biologically active agent include anti-cancer agent, immunosuppressant, antibiotic, antirheumatic agent, antithrombotic agent, HMG-CoA reductase inhibitor, ACE inhibitor, calcium antagonist, antilipemic agent, anti-inflammatory drug, integrin inhibitor, anti-allergic agent, antioxidant agent, GPIIbIIIa antagonist, retinoid, flavonoid, carotenoid, lipid-improving agent, DNA synthesis inhibitor, tyrosine kinase inhibitor, antiplatelet agent, proliferation inhibitor for vascular smooth muscle cell, anti-inflammatory agent, material of biological origin, interferon, and NO production enhancing substance.

As the anti-cancer agent, e.g. the following substances are preferable: vincristine, vinblastine, vindesine, irinotecan, pirarubicin, paclitaxel, docetaxel, and methotrexate.

As the immunosuppressant, e.g. the following substances are preferable: sirolimus, sirolimus derivatives such as everolimus, pimecrolimus, ABT-578, AP23573, and CCI-779, tacrolimus, azathioprine, ciclosporin, cyclophosphamide, mycophenolate mofetil, gusperimus, and mizoribine.

As the antibiotic, e.g. the following substances are preferable: mitomycin, adriamycin, doxorubicin, actinomycin, daunorubicin, idarubicin, pirarubicin, aclarubicin, epirubicin, peplomycin, and zinostatin stimalamer.

As the antirheumatic agent, e.g. methotrexate, sodium thiomalate, penicillamine, and lobenzarit are preferable.

As the antithrombotic agent, e.g. heparin, aspirin, anti-thrombin drug, ticlopidine, and hirudin are preferable.

As the HMG-CoA reductase inhibitor, e.g. the following substances are preferable: cerivastatin, cerivastatin sodium, atorvastatin, rosuvastatin, pitavastatin, fluvastatin, fluvastatin sodium, simvastatin, lovastatin, and pravastatin.

As the ACE inhibitor, e.g. the following substances are preferable: quinapril, perindopril erbumine, trandolapril, cilazapril, temocapril, delapril, enalapril maleate, lisinopril, and captopril.

As the calcium antagonist, e.g. nifedipine, nilvadipine, diltiazem, benidipine, and nisoldipine are preferable.

As the antilipemic agent, e.g. probucol is preferable.

As the integrin inhibitor, e.g. AJM300 is preferable.

As the anti-allergic agent, e.g. tranilast is preferable.

As the antioxidant agent, e.g. a-tocopherol is preferable.

As the GPIIbIIIa antagonist, e.g. abciximab is preferable.

As the retinoid, e.g. all-trans retinoic acid is preferable.

As the flavonoid, e.g. epigallocatechin, anthocyanin, and proanthocyanidin are preferable.

As the carotenoid, e.g. β-carotene and lycopene are preferable.

As the lipid-improving agent, e.g. eicosapentaenoic acid is preferable.

As the DNA synthesis inhibitor, e.g. 5-FU is preferable.

As the tyrosine kinase inhibitor, e.g. genistein, tyrphostin, erbstatin, and staurosporine are preferable.

As the antiplatelet agent, e.g. ticlopidine, cilostazol, and clopidogrel are preferable.

As the anti-inflammatory agent, e.g. steroid such as dexamethasone and prednisolone is preferable.

As the material of biological origin, e.g. the following substances are preferable: EGF (epidermal growth factor), VEGF (vascular endothelial growth factor), HGF (hepatocyte growth factor), PDGF (platelet derived growth factor), and BFGF (basic fibroblast growth factor).

As the interferon, e.g. interferon-γ1a is preferable.

As the NO production enhancing substance, e.g. L-arginine is preferable.

Whether only one kind of biologically active agent is employed or two or more kinds of different biologically active agents are combined should be arbitrarily selected according to the specifics of a case.

As one example of the manufacturing method of the protrusions 132 on the projecting bases 130, a method for manufacturing the protrusions 132 made of a resin will be described.

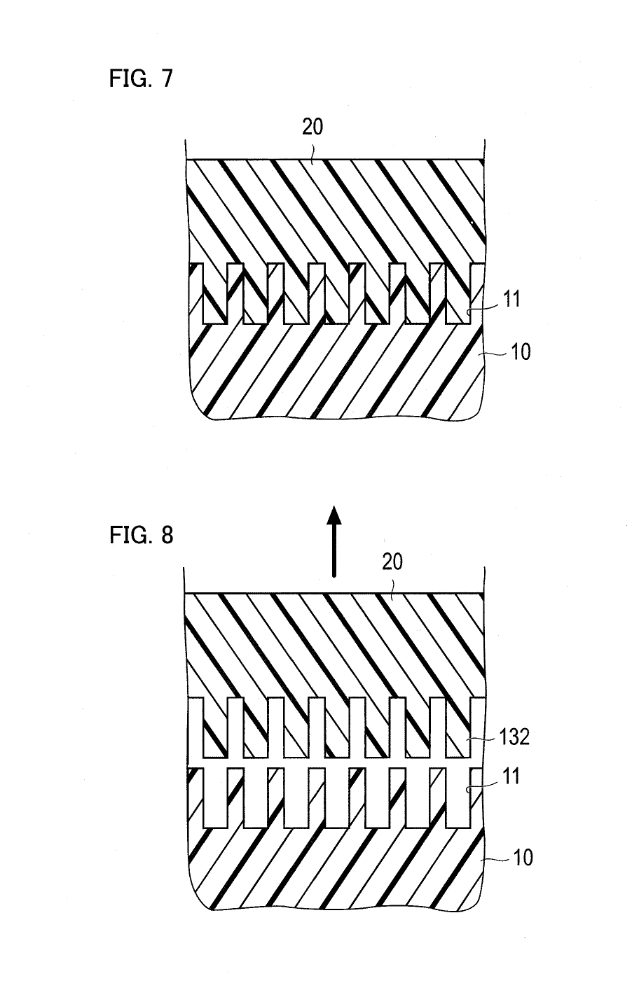

First, a mold 10 is fabricated by forming a micro-pattern 11 with a shape of holes on the order of several hundreds of nanometers in a poly(methyl methacrylate) (PMMA) resin supported on a silicon wafer by electron beam lithography (see FIG. 6). The shape of the micro-pattern 11 is decided so as to correspond with the shape obtained by transferring the protrusions 132 to be fabricated on the protrusion-formed surface 131.

Next, as the material of the protrusions 132, the above-described resin material is dissolved in a liquid at 0.001 to 1 wt % to be turned to a sol phase. As to the liquid, chloroform is exemplary of a liquid that can be used.

The surface of the mold 10 in which the micro-pattern 11 is formed is oriented upward and made horizontal. Then, as shown in FIG. 7, the material turned to the sol phase is poured into this mold 10 to make the material infiltrate the micro-pattern 11, and the material is further poured to obtain a predetermined thickness. Thereafter, the mold 10 is heated to a room temperature to 40° C. to volatilize the liquid and coagulate the material. (When using a thermoplastic material, the material is poured into the mold 10 after being heated to be melted, and then is cooled to be coagulated.)

After the material is coagulated, the coagulated material is removed from the mold 10 as shown in FIG. 8, so that a sheet 20 on which the plural protrusions 132 are formed is obtained. Thereafter, the sheet 20 is bonded onto the projecting base 130 of the substrate 110 (fabricated in a different step), which provides a configuration in which the protrusions 132 are provided on the protrusion-formed surface 131.

In the exemplary technique described above, the shape of the protrusion 132 is not limited as long as it is such a shape as to allow release from the mold 10. For example, a conical shape or a pyramidal shape can also be employed. Furthermore, although the sheet 20 on which the protrusions 132 are formed is attached to the projecting base 130 in the exemplary embodiment, it is also possible to monolithically form the projecting base 130 and the substrate 110 in a mold, simultaneously with the formation of the protrusions 132.

With respect to the processing of the pattern on the order of several hundreds of nanometers, not only can the above-described method be utilized, but also, for example, nanoimprinting, soft lithography, and shaping by use of a minute bit (e.g. diamond bit) can be applied. It is preferable to properly select the method depending on the conditions such as the shape, dimensions, material, etc. of the protrusion 132. When having a pyramidal shape, the protrusions 132 can easily be fabricated by forming grooves vertically and horizontally by a minute bit.

A device 200 for attaching the above-described biological adhesive sheet 100 to the living tissue M will now be described.

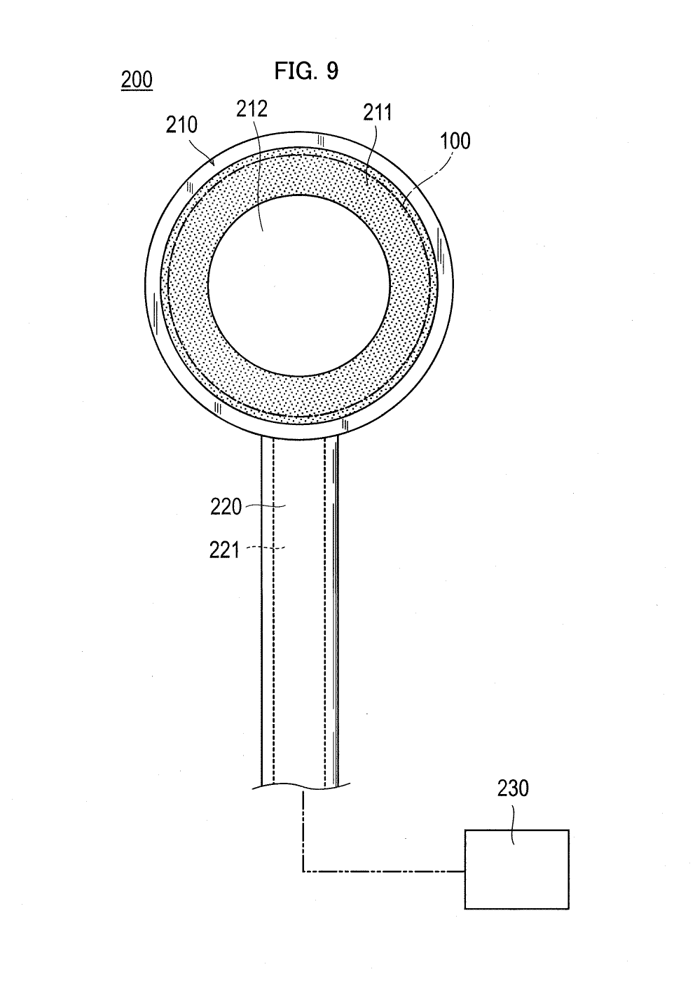

As shown in FIG. 9, the device for attaching a biological adhesive sheet 200 includes a holding section 210 that holds the biological adhesive sheet 100 and an operation section 220 joined to the holding section 210 for operating the holding section 210. The holding section 210 can be operated so that the operator grasps one side of the operation section 220 opposite to the side joined to the holding section 210.

In the holding section 210, a suction part 211 that can suction the biological adhesive sheet 100 by generating a negative pressure and a non-suction part 212 that does not generate a negative pressure and does not have suction power are formed. Plural minute holes are formed in the suction part 211. The suction part 211 is supplied with a negative pressure from an external negative pressure supply source 230 through a negative pressure supply path 221 passing through the inside of the operation section 220. This allows the suction part 211 to absorb the biological adhesive sheet 100 in contact with the holding section 210 and absorb the living tissue M through the through-holes 111 of the biological adhesive sheet 100. The suction part 211 is formed into an annular shape corresponding to the adhesive section 120 in which the through-holes 111 of the biological adhesive sheet 100 are formed. The non-suction part 212 is formed so as to be surrounded by the suction part 211. The form of the suction part 211 and the non-suction part 212 is not limited to the above-described configuration. It is not particularly limited as long as the biological adhesive sheet 100 can be suctioned and held to the holding section 210 and the living tissue M can be drawn through the through-holes 111 of the biological adhesive sheet 100. Therefore, for example the non-suction part 212 does not need to be provided and the suction part 211 does not need to be formed into an annular shape. Furthermore, the suction part 211 may be formed so as to be divided into plural areas.

The constituent material of the holding section 210 is not particularly limited as long as the biological adhesive sheet 100 can be held and the living tissue M can be absorbed. For example, a thermoplastic resin, such as a general plastic, or a thermosetting resin or a thermally crosslinkable resin, such as rubber, can all be used as the material. Specific examples of the material include the following various kinds of thermoplastic resins and polymer derivatives thereof: polyesters such as polyethylene terephthalate and polybutylene terephthalate and polyester elastomers containing them as a hard segment, polyolefins such as polyethylene and polypropylene and polyolefin elastomers, copolymer polyolefin using a metallocene catalyst, vinyl-based polymers such as polyvinyl chloride, PVDC, and PVDF, polyamide containing nylon and polyamide elastomer (PAE), polyimide, polystyrene, SEBS resin, polyurethane, polyurethane elastomer, ABS resin, acrylic resin, polyarylate, polycarbonate, polyoxymethylene (POM), polyvinyl alcohol (PVA), fluorine resin (ETFE, PFA, PTFE), saponified ethylene-vinyl acetate, ethylene-copoly-vinyl alcohol, ethylene vinyl acetate, carboxymethylcellulose, methylcellulose, cellulose acetate, poly(vinyl sulfone), liquid crystal polymer (LCP), polyethersulfone (PES), polyetheretherketone (PEEK), polyphenylene oxide (PPO), and polyphenylene sulfide (PPS). In addition, the specific examples of the material include the following thermosetting or thermally crosslinkable resins: vulcanized rubber, silicon-based resins such as polydimethylsiloxane (PMDS) and polyvinylsilane (PVS), epoxy resin, and two-component reactive polyurethane resin. Moreover, a polymer alloy containing any of the above-described thermoplastic resins and thermosetting or thermally crosslinkable resins can also be used, and a resin solution obtained by dissolving a resin in a liquid may be used as the forming material.

The holding section 210 may also be capable of being flexibly deformed along the living tissue M when the biological adhesive sheet 100 is brought into contact with the living tissue M.

A method for attaching the biological adhesive sheet 100 to the living tissue M by the device for attaching the biological adhesive sheet 200 will be described below.

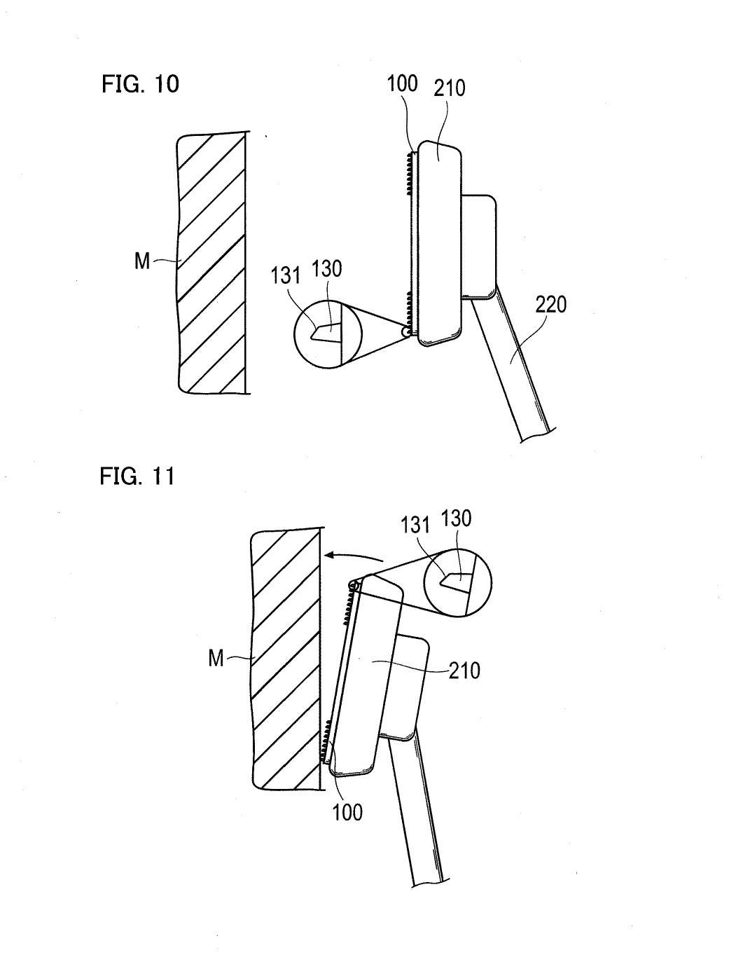

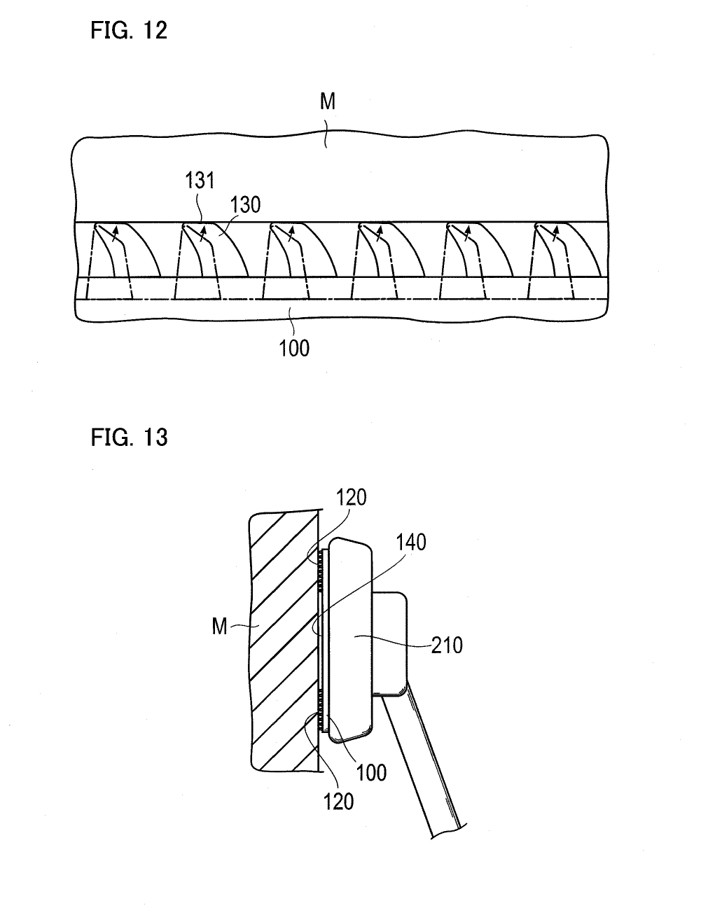

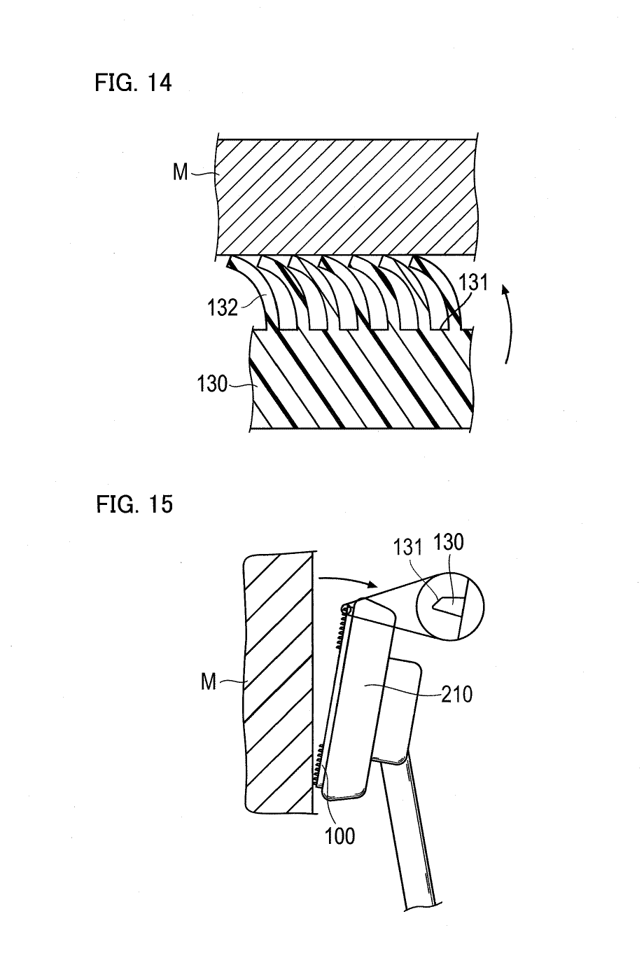

First, with supply of a negative pressure to the suction part 211 of the device for attaching the biological adhesive sheet 200 by the negative pressure supply source 230, the biological adhesive sheet 100 is brought close to the holding section 210 and the surface of the biological adhesive sheet 100 on the opposite side to the surface in which the adhesive section 120 is formed is absorbed and held by the holding section 210. Next, as shown in FIG. 10, the holding section 210 is operated with the operation section 220 grasped, and the holding section 210 of the device for attaching the biological adhesive sheet 200 is brought close to the living tissue M. Then, as shown in FIG. 11, the held biological adhesive sheet 100 is brought close to the living tissue M from the higher side of the protrusion-formed surfaces 131. The biological adhesive sheet 100 does not need to be pressed against the living tissue M. At this time, the suction part 211 draws the living tissue M through the through-holes 111. Thus, as shown in FIG. 12, the projecting bases 130 are deformed and the protrusion-formed surfaces 131 formed with an inclination make contact with the living tissue M. Because the plural protrusions 132 are formed in the adhesive section 120, the adhesive section 120 adheres to the living tissue M and is held by the Van der Waals' forces as shown in FIGS. 13 and 14. Based on this, for example if the living tissue M is a lung in which an air leakage part is formed in pneumothorax, the adhesive section 120 of the biological adhesive sheet 100 adheres to tissue around the air leakage part and the flat surface section 140 covers the air leakage part. This can suppress leakage of inspired air from the air leakage part to a lung cavity. Furthermore, for example, if the living tissue M is the left ventricle in the case of dilated cardiomyopathy, the stretch of the myocardium can be physically suppressed by the biological adhesive sheet 100. In the case of using the biological adhesive sheet 100 for the left ventricle in a treatment for dilated cardiomyopathy, the stretch of the myocardium can be suppressed more effectively by forming the adhesive section 120 in an entirety of the biological adhesive sheet 100, that is, without forming the flat surface section 140 having no adhesive force in the biological adhesive sheet 100.

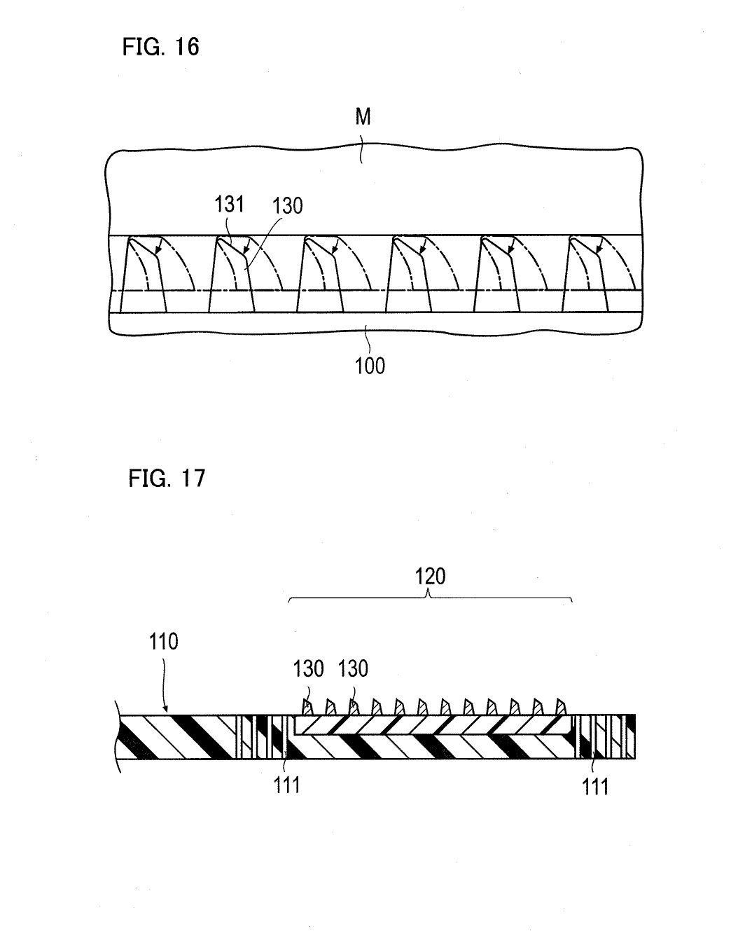

Once the biological adhesive sheet 100 is attached and it is desired to peel off the biological adhesion sheet 100 and attach it again, the biological adhesion sheet 100 is peeled off from one side (lower side of the protrusion-formed surfaces 131). Thus, as shown in FIGS. 15 and 16, the protrusion-formed surfaces 131 of the projecting bases 130 are separated from only one direction and the biological adhesive sheet 100 can be peeled off with as little a load as possible being imposed on the living tissue M.

Thereafter, when the supply of the negative pressure by the negative pressure supply source 230 is stopped, the absorption force of the suction part 211 is lost and the biological adhesive sheet 100 detaches from the holding section 210. The biological adhesive sheet 100 is maintained so as to still stick to the living tissue M. The device for attaching the biological adhesive sheet 200 is then separated from the living tissue M so that the procedure is completed.

In the biological adhesive sheet 100 according to the exemplary embodiment, the protrusions 132 make contact with the living tissue M and adhere thereto by the Van der Waals' forces by drawing the living tissue M from the other surface side of the substrate 110 through the through-holes 111. Therefore, a strong adhesive force can be exerted almost without requiring a pressing force when the biological adhesive sheet 100 is attached to the living tissue M, and safety is enhanced.

Furthermore, one or more protrusions 132 are formed per 100 μm² and the protrusions 132 have a length of 1 μm to 50 μm and a maximum outer diameter of 5 nm to 10 μm. Therefore, in either a gas or a liquid, the protrusions 132 can be made to favorably adhere to the living tissue M by utilizing the Van der Waals' forces.

In addition, the adhesive section 120 can be partially formed on the substrate 110. Thus, it can be variously designed depending on the application site of the living tissue M and the purpose of use.

Moreover, the adhesive section 120 includes the projecting bases 130, which are formed so as to protrude from the outer surface of the substrate 110 and each have the protrusion-formed surface 131 inclined relative to the outer surface, and the protrusions 132 are formed on the protrusion-formed surface 131. Therefore, when the inclined protrusion-formed surfaces 131 are separated from the living tissue M, they get detached from one side. The protrusions 132 formed on the protrusion-formed surface 131 can thus be easily detached from the living tissue M. Furthermore, due to the forming of the protrusions 132 on the inclined protrusion-formed surface 131, when the adhesive section 120 is brought into contact with the living tissue M, the adhesive section 120 readily makes contact with the living tissue M from one side of the protrusion-formed surface 131. This enhances ease in handling.

In addition, because the porosity of the through-holes 111 is 45% to 85%, the living tissue M can be favorably drawn (suctioned) through the through-holes 111.

The device for attaching the biological adhesive sheet 200 according to the exemplary embodiment suctions the living tissue M with the suction part 211 to draw the living tissue M through the through-holes 111 of the biological adhesive sheet 100. The device 200 thus brings the adhesive section 120 of the biological adhesive sheet 100 into contact with the living tissue M and makes it adhere thereto. The biological adhesive sheet 100 can thereby be made to strongly adhere to the living tissue M almost without requiring a pressing force when being attached thereto, and safety is enhanced.

In addition, the suction part 211 is partially formed at the site in the holding section 210 with which the biological adhesive sheet 100 makes contact. Thus, only the site requiring suction can be suctioned depending on the application site of the living tissue M and the purpose of use.

The disclosure herein is not limited to the above-described exemplary embodiments and various changes can be made by those skilled in the art in the technical idea of the disclosure. For example, the arrangement of the flat surface section 140 having no adhesive force and the adhesive section 120 in the biological adhesive sheet 100 is not limited to the above-described configuration and it is preferable to properly change the arrangement depending on the application site of the living tissue M and the purpose for use. Therefore, for example it is also possible that the adhesive section 120 is formed so as to be scattered on the substrate 110. Furthermore, the shape of the biological adhesive sheet 100 is not limited to the circular shape and it is preferable to properly change the shape depending on the application site of the living tissue M and the purpose for use.

Other configurations, such as the exemplary modification shown in FIG. 17 can also be employed. Specifically, through-holes are not formed in the adhesive section 120, but rather, the through-holes 111 are formed in the substrate 110 surrounding the adhesive section 120. The adhesive section 120 is brought into contact with living tissue by applying a suction force from the periphery of the adhesive section 120.

In addition, the protrusion-formed surface 131 of the projecting base 130 does not necessarily need to be inclined to the outer surface of the substrate 110. The protrusions 132 may be formed directly on the substrate 110 without forming the projecting base 130. The extension direction of the minute protrusions 132 may be irregular.

Furthermore, when the biological adhesive sheet 100 is attached, it does not necessarily need to be attached so as to be brought into contact from one side as long as adhesion of the adhesive section 120 can be made.

The detailed description above describes a biological adhesive sheet and device for attaching a biological adhesive sheet by way of example. The invention is not limited, however, to the precise embodiment and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A biological adhesive sheet comprising:
a substrate including a plurality of through-holes formed therein, the substrate including an adhesive section having a plurality of protrusions that protrude from one side surface of the substrate;
wherein the protrusions are configured to make contact with a living tissue and to adhere to the living tissue by virtue of a Van der Waals' forces when the living tissue is suctioned through the through-holes from an opposing side surface of the substrate.

2. The biological adhesive sheet according to claim 1, wherein
the plurality of protrusions includes at least one per 100 μm² of the adhesive section and the protrusions have a length of 1 μm to 50 μm and a maximum outer diameter of 5 nm to 10 μm.

3. The biological adhesive sheet according to claim 1, wherein
the adhesive section is partially formed on the substrate.

4. The biological adhesive sheet according to claim 1, wherein
the adhesive section has a projecting base that is formed so as to protrude from an outer surface of the substrate and a protrusion-formed surface inclined relative to the outer surface of the substrate, and
the protrusions are formed on the protrusion-formed surface.

5. The biological adhesive sheet according to claim 1, wherein
a porosity of the through-holes is 45% to 85%.

6. A device for attaching a biological adhesive sheet comprising:
a holding section that holds a biological adhesive sheet in which a plurality of through-holes are formed and that is configured to bring an adhesive section of the biological adhesive sheet into contact with living tissue, wherein
the holding section has a suction part supplied with a negative pressure allowing suction of the living tissue through the through-holes of the biological adhesive sheet, and
the suction part is configured to suction the living tissue through the through-holes of the biological adhesive sheet to bring the adhesive section of the biological adhesive sheet into contact with the living tissue such that the adhesive section adheres to the living tissue.

7. The device for attaching a biological adhesive sheet according to claim 6, wherein
the suction part is partially formed at a site in the holding section with which the biological adhesive sheet makes contact.

8. A biological adhesive sheet comprising:
a substrate; and
an adhesive section disposed on the substrate;
wherein the adhesive section is configured to exert an adhesive force on a living tissue by virtue of Van der Waals' forces.

9. The biological adhesive sheet according to claim 8, wherein the adhesive section includes a plurality of minute protrusions.

10. The biological adhesive sheet according to claim 9, wherein the adhesive section includes a plurality of projecting bases protruding from a first surface of the substrate.

11. The biological adhesive sheet according to claim 10, wherein each of the projecting bases includes an inclined surface, said plurality of minute protrusions protruding from the inclined surface of each of the projecting bases.

12. The biological adhesive sheet according to claim 11, wherein Van der Waals' forces are generated between said plurality of minute protrusions and the living tissue when the adhesive section is brought into contact with the living tissue.

13. The biological adhesive sheet according to claim 12, wherein the adhesive section of the substrate includes a plurality of through-holes.

14. The biological adhesive sheet according to claim 13, wherein, when suction is applied from an opposing, second surface of the substrate, living tissue is drawn through the through-holes of the biological adhesive sheet to bring the adhesive section of the biological adhesive sheet into contact with living tissue such that the adhesive section adheres to the living tissue.

15. A system for attaching a biological adhesive sheet to a living tissue comprising:
a biological adhesive sheet including a first surface having an adhesive section having a plurality of through-holes and a second surface opposite thereto;
a holding section configured to hold the biological adhesive sheet, the holding section including a suction part supplied with a negative pressure; and
wherein, when the second surface of the biological adhesive sheet is brought close to the holding section and negative pressure is supplied to the suction part, the second surface of the biological adhesive sheet is held by the holding section;
wherein, when the holding section is brought close to a living tissue and negative pressure is supplied to the suction part, the living tissue is suctioned through the through-holes of the biological adhesive sheet so as to bring the first surface of the biological adhesive sheet having the adhesive section into contact with the living tissue, whereby the adhesive section adheres to the living tissue.

16. The system according to claim 15, wherein the adhesive section is configured to exert an adhesive force on the living tissue by virtue of Van der Waals' forces.

17. The system according to claim 16, wherein the adhesive section includes a plurality of minute protrusions.

18. The system according to claim 17, wherein the adhesive section includes a plurality of projecting bases protruding from the first surface thereof.

19. The system according to claim 18, wherein each of the projecting bases includes an inclined surface, said plurality of minute protrusions protruding from the inclined surface of each of the projecting bases.

20. The system according to claim 19, wherein Van der Waals' forces are generated between said plurality of minute protrusions and the living tissue when the adhesive section is brought into contact with the living tissue.
